(19) United States
(12) Patent Application Publication
(10) Pub. No.: US 2022/0241364 A1
(43) Pub. Date: Aug. 4, 2022

(54) Composition for Lowering Blood Glucose Level, Reducing Blood Hemoglobin A1c Content, or Increasing Blood HDL-Cholesterol Content

(71) Applicant: House Wellness Foods Corporation, Hyogo (JP)

(72) Inventors: Ryusei Uchio, Itami-shi, Hyogo (JP); Koutarou Muroyama, Itami-shi, Hyogo (JP); Kengo Kawasaki, Itami-shi, Hyogo (JP); Shinji Murosaki, Itami-shi, Hyogo (JP)

(73) Assignee: House Wellness Foods Corporation, Hyogo (JP)

(21) Appl. No.: 17/624,530

(22) PCT Filed: Jun. 29, 2020
(86) PCT No.: PCT/JP2020/025571

§ 371 Date: Jan. 3, 2022

(30) Foreign Application Priority Data
Jul. 1, 2019 (JP) 2019-122895

(51) Int. Cl. A61K 36/9066; A61K 31/12

(52) U.S. Cl. CPC A61K 36/9066; A61K 31/12

(57) ABSTRACT

One or more embodiments of the present invention relate to a composition for improving lipid metabolism or carbohydrate metabolism, comprising a component derived from turmeric. One or more embodiments of the present invention relate to a composition for lowering blood glucose level, reducing blood hemoglobin A1c content, or increasing blood HDL-cholesterol content, a composition for treating or preventing metabolic syndrome, or a composition for improving lipid metabolism or carbohydrate metabolism, wherein each composition comprises, as an active ingredient, at least one of turmeronol A, turmeronol B, and bisacurone. Each composition may comprise 20 μg or more of turmeronol A and turmeronol B in total per daily intake, may comprise 17 μg or more of turmeronol A per daily intake, may comprise 5 μg or more of turmeronol B per daily intake, and may comprise 80 μg or more of bisacurone per daily intake.

### TECHNICAL FIELD

One or more embodiments of the present invention relate to a composition for lowering blood glucose level, reducing blood hemoglobin A1c content, or increasing blood HDL-cholesterol content.

Further, one or more embodiments of the present invention relate to a composition for treating or preventing metabolic syndrome.

Furthermore, one or more embodiments of the present invention relate to a composition for improving lipid metabolism or carbohydrate metabolism.

### BACKGROUND ART

Blood sugar and hemoglobin A1c (HbA1c) are indexes used for diagnosis of diabetes mellitus. Increased concentrations thereof in blood are believed to be one of causes for disorder of microvessels or great vessels, and known to increase the risk of onset of various diabetic complications (Non Patent Literature 1).

High-density lipoprotein (HDL) cholesterol plays a role for conveying cholesterols present in blood or tissue to the liver, and prevents cholesterols from accumulating in the periphery. The onset of low HDL cholesterolemia leads to accumulation of cholesterols in blood vessels, increasing the risk of arteriosclerotic diseases (Non Patent Literature 2).

Turmeric extracts are known to have various bioactivities.

Patent Literature 1 discloses that an extract of a turmeric rhizome extracted with 50% methanol aqueous solution comprises a curcumin derivative, and that a fraction obtained by fractionation of the extract has α-amylase inhibitory activity and is effective for suppressing hyperglycemia or the like.

Patent Literature 2 discloses that rats fed with a high-fat/high-cholesterol diet for 8 weeks underwent reduction in blood cholesterol after being grown with oral administration of a test diet containing turmeric blended with 16 additional plant materials. Patent Literature 2 further discloses that five volunteers who had taken the same test diet for 3 months were found to have a tendency of lower fasting blood sugar level.

Patent Literature 3 discloses that an ethanol extract of turmeric orally ingested by mice provided with a high-fat/high-sugar diet inhibited the increase of intraperitoneal fat weight and blood sugar level in the mice.

Patent Literature 4 discloses that essential oil components of turmeric, including ar turmerone, have an effect of inhibiting the elevation of blood sugar level.

Patent Literature 5 discloses that when being administered to rats, a composition comprising a dry powder of turmeric and a plurality of additional herbal medicine components inhibited the increase of blood sugar level in the rats, and that humans who had drunk the compound underwent the lowering of blood sugar level and hemoglobin A1c (HbA1c) level.

Non Patent Literature 3 discloses that, in a diabetic and dyslipidemic model test with rats, administration of 300 mg/mL of a methanol extract of turmeric by drinking with water for 42 days significantly improved fasting blood sugar level and blood lipid (TG, total cholesterol, LDL, HDL).

Non Patent Literature 4 discloses that when being administered to a diabetic rat model, a composition comprising a turmeric hot-water extract in combination with additional herbal medicine components significantly lowered fasting blood sugar level and reduced the amount of blood sialic acid, and significantly improved blood lipid in the model.

Non Patent Literature 5 discloses that when being administered to rats for which diabetes mellitus had been induced, a combination of a water extract of turmeric and a water extract of Abroma augusta significantly reduced blood glucose and significantly increased total hemoglobin content in the rats. Non Patent Literature 5 discloses that the active component of the water extract of turmeric is curcumin.

Non Patent Literature 6 discloses that, in a hypercholesterolemia/fatty liver rat model, a turmeric extract reduced the amounts of total cholesterol and LDL in blood and increased the amount of HDL in blood.

Non Patent Literature 7 discloses that when being fed to rats for which hyperlipidemia had been induced with an agent, a 50% ethanol extract of turmeric increased the HDL/total cholesterol ratio and decreased the total cholesterol/phospholipid ratio in the rats.

### CITATION LIST

### Patent Literature

Patent Literature 1: JP Patent Publication (Kokai) No. 2014-031330 A
Patent Literature 2: JP Patent Publication (Kokai) No. 2010-202634 A
Patent Literature 3: JP Patent Publication (Kokai) No. 2014-177497 A
Patent Literature 4: WO 2005/115366
Patent Literature 5: JP Patent Publication (Kokai) No. 2005-325025 A

### Non Patent Literature

Non Patent Literature 1: The Japan Diabetes Society (2013). “Evidence-based Practice Guideline for the Treatment for Diabetes in Japan 2013”, Nankodo Co., Ltd.
Non Patent Literature 2: Japan Atherosclerosis Society (2017). “Japan Atherosclerosis Society (JAS) Guidelines for Prevention of Atherosclerotic Cardiovascular Diseases 2017”, Kowa Insatsu K.K.
Non Patent Literature 3: Pak. J. Pharm. Sci., 2018 March; 31(2): 491-498
Non Patent Literature 4: Chin. J. Integr. Med., 2016 Apr. 27
Non Patent Literature 5: Indian J. Clin. Biochem., 2002, 17(2): 33-43
Non Patent Literature 6: J. Food Sci. 2011 April; 76(3): H80-9
Non Patent Literature 7: Indian J. Physiol. Pharmacol., 1988, 32(4): 299-304
Non Patent Literature 8: Today's Laboratory Test (original title in Japanese: Konnichi no Rinsho-Kensa) 2015-2016, 14th ed. (Nankodo Co., Ltd.), pp. 142-143, 154-155

### SUMMARY OF INVENTION

### Technical Problem

An object of one or more embodiments of the present invention is to provide a composition effective for lowering blood glucose level, reducing blood hemoglobin A1c content, or increasing blood HDL-cholesterol content. Further, an object of one or more embodiments of the present invention is to provide a composition for treating or preventing metabolic syndrome. Furthermore, an object of one or more embodiments of the present invention is to provide a composition for improving lipid metabolism or carbohydrate metabolism.

### Solution to Problem

The present inventors have found that blood glucose level was significantly lowered, blood hemoglobin A1c content significantly reduced, and blood HDL-cholesterol content significantly increased in humans who had ingested tablets each comprising a turmeric extract comprising turmeronol A, turmeronol B, and bisacurone every day for 12 weeks. This has led to the completion of one or more embodiments of the present invention described below.

(1) A composition for lowering blood glucose level, reducing blood hemoglobin A1c content, or increasing blood HDL-cholesterol content, wherein the composition comprises at least one of turmeronol A, turmeronol B, and bisacurone.
(2) The composition for lowering blood glucose level, reducing blood hemoglobin A1c content, or increasing blood HDL-cholesterol content according to (1), wherein the composition comprises a turmeric extract comprising the at least one of turmeronol A, turmeronol B, and bisacurone and extracted with water.
(3) A composition for lowering blood glucose level, reducing blood hemoglobin A1c content, or increasing blood HDL-cholesterol content, wherein the composition comprises a turmeric extract comprising at least one of turmeronol A, turmeronol B, and bisacurone and extracted with water.
(4) The composition for lowering blood glucose level, reducing blood hemoglobin A1c content, or increasing blood HDL-cholesterol content according to any one of (1) to (3), wherein the composition comprises 20 μg or more of turmeronol A and turmeronol B in total per daily intake.
(5) The composition for lowering blood glucose level, reducing blood hemoglobin A1c content, or increasing blood HDL-cholesterol content according to any one of (1) to (4), wherein the composition comprises 17 μg or more of turmeronol A per daily intake.
(6) The composition for lowering blood glucose level, reducing blood hemoglobin A1c content, or increasing blood HDL-cholesterol content according to any one of (1) to (5), wherein the composition comprises 5 μg or more of turmeronol B per daily intake.
(7) The composition for lowering blood glucose level, reducing blood hemoglobin A1c content, or increasing blood HDL-cholesterol content according to any one of (1) to (6), wherein the composition comprises 80 μg or more of bisacurone per daily intake.
(8) The composition for lowering blood glucose level, reducing blood hemoglobin A1c content, or increasing blood HDL-cholesterol content according to any one of (1) to (7), wherein the content of curcumin is less than 30 mg per daily intake.
(9) The composition for lowering blood glucose level, reducing blood hemoglobin A1c content, or increasing blood HDL-cholesterol content according to any one of (1) to (8), for administering to a healthy human.
(10) A composition for treating or preventing metabolic syndrome, comprising at least one of turmeronol A, turmeronol B, and bisacurone.
(11) A composition for treating or preventing metabolic syndrome, comprising a turmeric extract comprising at least one of turmeronol A, turmeronol B, and bisacurone and extracted with water.
(12) A composition for improving lipid metabolism or carbohydrate metabolism, comprising at least one of turmeronol A, turmeronol B, and bisacurone.
(13) A composition for improving lipid metabolism or carbohydrate metabolism, comprising a turmeric extract comprising at least one of turmeronol A, turmeronol B, and bisacurone and extracted with water.
(14) The composition according to any one of (10) to (13), wherein the composition comprises 20 μg or more of turmeronol A and turmeronol B in total per daily intake.
(15) The composition according to any one of (10) to (14), wherein the composition comprises 17 μg or more of turmeronol A per daily intake.
(16) The composition according to any one of (10) to (15), wherein the composition comprises 5 μg or more of turmeronol B per daily intake.
(17) The composition according to any one of (10) to (16), wherein the composition comprises 80 μg or more of bisacurone per daily intake.
(18) The composition according to any one of (10) to (17), wherein the content of curcumin is less than 30 mg per daily intake.
(19) The composition according to any one of (10) to (18), for administering to a healthy human.
(20) Use of a at least one of turmeronol A, turmeronol B, and bisacurone, or a turmeric extract comprising at least one of turmeronol A, turmeronol B, and bisacurone and extracted with water, for production of a composition for lowering blood glucose level, reducing blood hemoglobin A1c content, or increasing blood HDL-cholesterol content.
(21) Use of a at least one of turmeronol A, turmeronol B, and bisacurone, or a turmeric extract comprising at least one of turmeronol A, turmeronol B, and bisacurone and extracted with water, for production of a medicament for lowering blood glucose level, reducing blood hemoglobin A1c content, or increasing blood HDL-cholesterol content.

In (20) or (21), the composition or medicament is preferably a composition or medicament for treating or preventing a disease or symptom that is ameliorated by lowering blood glucose level, reducing blood hemoglobin A1c content, or increasing blood HDL-cholesterol content.

(22) The use according to (20) or (21), wherein the composition or medicament comprises 20 μg or more of turmeronol A and turmeronol B in total per daily intake.
(23) The use according to any one of (20) to (22), wherein the composition or medicament comprises 17 μg or more of turmeronol A per daily intake.
(24) The use according to any one of (20) to (23), wherein the composition or medicament comprises 5 μg or more of turmeronol B per daily intake.
(25) The use according to any one of (20) to (24), wherein the composition or medicament comprises 80 μg or more of bisacurone per daily intake.
(26) The use according to any one of (20) to (25), wherein the content of curcumin in the composition or medicament is less than 30 mg per daily intake.
(27) The use according to any one of (20) to (26), wherein the composition or medicament is for administering to a healthy human.
(28) A method for lowering blood glucose level, reducing blood hemoglobin A1c content, or increasing blood HDL-cholesterol content, comprising:

administering at least one of turmeronol A, turmeronol B, and bisacurone, or a turmeric extract comprising at least one of turmeronol A, turmeronol B, and bisacurone and extracted with water, to a subject; and

lowering blood glucose level, reducing blood hemoglobin A1c content, or increasing blood HDL-cholesterol content in the subject.

(29) The method according to (28), wherein the subject is a subject in need of lowering blood glucose level, reducing blood hemoglobin A1c content, or increasing blood HDL-cholesterol content or for whom lowering blood glucose level, reducing blood hemoglobin A1c content, or increasing blood HDL-cholesterol content is desirable.
(30) The method according to (28) or (29), wherein the subject is a healthy human.
(31) The method according to any one of (28) to (30), wherein 20 μg or more of turmeronol A and turmeronol B in total is administered to the subject per day.
(32) The method according to any one of (28) to (31), wherein 17 μg or more of turmeronol A is administered to the subject per day.
(33) The method according to any one of (28) to (32), wherein 5 μg or more of turmeronol B is administered to the subject per day.
(34) The method according to any one of (28) to (33), wherein 80 μg or more of bisacurone is administered to the subject per day.
(35) The method according to any one of (28) to (34), wherein the daily dose of curcumin is less than 30 mg.
(36) At least one of turmeronol A, turmeronol B, and bisacurone, or a turmeric extract comprising at least one of turmeronol A, turmeronol B, and bisacurone and extracted with water, for lowering blood glucose level, reducing blood hemoglobin A1c content, or increasing blood HDL-cholesterol content in a subject.
(37) The compound or turmeric extract according to (36), wherein the subject is a subject in need of lowering blood glucose level, reducing blood hemoglobin A1c content, or increasing blood HDL-cholesterol content or for whom lowering blood glucose level, reducing blood hemoglobin A1c content, or increasing blood HDL-cholesterol content is desirable.
(38) The compound or turmeric extract according to (36) or (37), wherein the subject is a healthy human.
(39) The compound or turmeric extract according to any one of (36) to (38), for lowering blood glucose level, reducing blood hemoglobin A1c content, or increasing blood HDL-cholesterol content by administering 20 μg or more of turmeronol A and turmeronol B in total to the subject per day.
(40) The compound or turmeric extract according to any one of (36) to (39), for lowering blood glucose level, reducing blood hemoglobin A1c content, or increasing blood HDL-cholesterol content by administering 17 μg or more of turmeronol A to the subject per day.
(41) The compound or turmeric extract according to any one of (36) to (40), for lowering blood glucose level, reducing blood hemoglobin A1c content, or increasing blood HDL-cholesterol content by administering 5 μg or more of turmeronol B to the subject per day.
(42) The compound or turmeric extract according to any one of (36) to (41), for lowering blood glucose level, reducing blood hemoglobin A1c content, or increasing blood HDL-cholesterol content by administering 80 μg or more of bisacurone to the subject per day.
(43) The compound or turmeric extract according to any one of (36) to (42), for lowering blood glucose level, reducing blood hemoglobin A1c content, or increasing blood HDL-cholesterol content by administering to the subject so that the daily dose of curcumin is less than 30 mg.
(44) Use of at least one of turmeronol A, turmeronol B, and bisacurone, or a turmeric extract comprising at least one of turmeronol A, turmeronol B, and bisacurone and extracted with water, for production of a composition for treating or preventing metabolic syndrome.
(45) Use of at least one of turmeronol A, turmeronol B, and bisacurone, or a turmeric extract comprising at least one of turmeronol A, turmeronol B, and bisacurone and extracted with water, for production of a medicament for treating or preventing metabolic syndrome.
(46) The use according to (44) or (45), wherein the composition or medicament comprises 20 μg or more of turmeronol A and turmeronol B in total per daily intake.
(47) The use according to any one of (44) to (46), wherein the composition or medicament comprises 17 μg or more of turmeronol A per daily intake.
(48) The use according to any one of (44) to (47), wherein the composition or medicament comprises 5 μg or more of turmeronol B per daily intake.
(49) The use according to any one of (44) to (48), wherein the composition or medicament comprises 80 μg or more of bisacurone per daily intake.
(50) The use according to any one of (44) to (49), wherein the content of curcumin in the composition or medicament is less than 30 mg per daily intake.
(51) The use according to any one of (44) to (50), wherein the composition or medicament is for administering to a healthy human.
(52) A method for treating or preventing metabolic syndrome, comprising:

administering at least one of turmeronol A, turmeronol B, and bisacurone, or a turmeric extract comprising at least one of turmeronol A, turmeronol B, and bisacurone and extracted with water, to a subject; and treating or preventing metabolic syndrome in the subject.

(53) The method according to (52), wherein the subject is a subject in need of treating or preventing metabolic syndrome or for whom treating or preventing metabolic syndrome is desirable.
(54) The method according to (52) or (53), wherein the subject is a healthy human.
(55) The method according to any one of (52) to (54), wherein 20 μg or more of turmeronol A and turmeronol B in total is administered to the subject per day.
(56) The method according to any one of (52) to (55), wherein 17 μg or more of turmeronol A is administered to the subject per day.
(57) The method according to any one of (52) to (56), wherein 5 μg or more of turmeronol B is administered to the subject per day.
(58) The method according to any one of (52) to (57), wherein 80 μg or more of bisacurone is administered to the subject per day.
(59) The method according to any one of (52) to (58), wherein the daily dose of curcumin is less than 30 mg.
(60) At least one of turmeronol A, turmeronol B, and bisacurone, or a turmeric extract comprising at least one of turmeronol A, turmeronol B, and bisacurone and extracted with water, for treating or preventing metabolic syndrome in a subject.
(61) The compound or turmeric extract according to (60), wherein the subject is a subject in need of treating or preventing metabolic syndrome or for whom treating or preventing metabolic syndrome is desirable.
(62) The compound or turmeric extract according to (60) or (61), wherein the subject is a healthy human.
(63) The compound or turmeric extract according to any one of (60) to (62), for treating or preventing metabolic syndrome by administering 20 μg or more of turmeronol A and turmeronol B in total to the subject per day.
(64) The compound or turmeric extract according to any one of (60) to (63), for treating or preventing metabolic syndrome by administering 17 μg or more of turmeronol A to the subject per day.
(65) The compound or turmeric extract according to any one of (60) to (64), for treating or preventing metabolic syndrome by administering 5 μg or more of turmeronol B to the subject per day.
(66) The compound or turmeric extract according to any one of (60) to (65), for treating or preventing metabolic syndrome by administering 80 μg or more of bisacurone to the subject per day.
(67) The compound or turmeric extract according to any one of (60) to (66), for treating or preventing metabolic syndrome by administering to the subject so that the daily dose of curcumin is less than 30 mg.
(68) Use of at least one of turmeronol A, turmeronol B, and bisacurone, or a turmeric extract comprising at least one of turmeronol A, turmeronol B, and bisacurone and extracted with water, for production of a composition for improving lipid metabolism or carbohydrate metabolism.
(69) Use of at least one of turmeronol A, turmeronol B, and bisacurone, or a turmeric extract comprising at least one of turmeronol A, turmeronol B, and bisacurone and extracted with water, for production of a medicament for improving lipid metabolism or carbohydrate metabolism.

In (68) or (69), the composition or medicament is preferably a composition or medicament for treating or preventing a disease or symptom that is ameliorated by improving lipid metabolism or carbohydrate metabolism.

(70) The use according to (68) or (69), wherein the composition or medicament comprises 20 μg or more of turmeronol A and turmeronol B in total per daily intake.
(71) The use according to any one of (68) to (70), wherein the composition or medicament comprises 17 μg or more of turmeronol A per daily intake.
(72) The use according to any one of (68) to (71), wherein the composition or medicament comprises 5 μg or more of turmeronol B per daily intake.
(73) The use according to any one of (68) to (72), wherein the composition or medicament comprises 80 μg or more of bisacurone per daily intake.
(74) The use according to any one of (68) to (73), wherein the content of curcumin in the composition or medicament is less than 30 mg per daily intake.
(75) A method for improving lipid metabolism or carbohydrate metabolism, comprising:

administering at least one of turmeronol A, turmeronol B, and bisacurone, or a turmeric extract comprising at least one of turmeronol A, turmeronol B, and bisacurone and extracted with water, to a subject; and

improving lipid metabolism or carbohydrate metabolism in the subject.

(76) The method according to (75), wherein the subject is a subject in need of improving lipid metabolism or carbohydrate metabolism or for whom improving lipid metabolism or carbohydrate metabolism is desirable.
(77) The method according to (75) or (76), wherein the subject is a healthy human.
(78) The method according to any one of (75) to (77), wherein 20 μg or more of turmeronol A and turmeronol B in total is administered to the subject per day.
(79) The method according to any one of (75) to (78), wherein 17 μg or more of turmeronol A is administered to the subject per day.
(80) The method according to any one of (75) to (79), wherein 5 μg or more of turmeronol B is administered to the subject per day.
(81) The method according to any one of (75) to (80), wherein 80 μg or more of bisacurone is administered to the subject per day.
(82) The method according to any one of (75) to (81), wherein the daily dose of curcumin is less than 30 mg.
(83) At least one of turmeronol A, turmeronol B, and bisacurone, or a turmeric extract comprising at least one of turmeronol A, turmeronol B, and bisacurone and extracted with water, for improving lipid metabolism or carbohydrate metabolism in a subject.
(84) The compound or turmeric extract according to (83), for improving lipid metabolism or carbohydrate metabolism by administering 20 μg or more of turmeronol A and turmeronol B in total to the subject per day.
(85) The compound or turmeric extract according to (83) or (84), for improving lipid metabolism or carbohydrate metabolism by administering 17 μg or more of turmeronol A to the subject per day.
(86) The compound or turmeric extract according to any one of (83) to (85), for improving lipid metabolism or carbohydrate metabolism by administering 5 μg or more of turmeronol B to the subject per day.
(87) The compound or turmeric extract according to any one of (83) to (86), for improving lipid metabolism or carbohydrate metabolism by administering 80 μg or more of bisacurone to the subject per day.
(88) The compound or turmeric extract according to any one of (83) to (87), for improving lipid metabolism or carbohydrate metabolism by administering to the subject so that the daily dose of curcumin is less than 30 mg.
(89) The compound or turmeric extract according to any one of (83) to (88), wherein the subject is a subject in need of improving lipid metabolism or carbohydrate metabolism or for whom improving lipid metabolism or carbohydrate metabolism is desirable.
(90) The compound or turmeric extract according to any one of (83) to (89), wherein the subject is a healthy human.
(91) The compound or turmeric extract according to any one of (83) to (90), for treating or preventing a disease or symptom that is ameliorated by improving lipid metabolism or carbohydrate metabolism in the subject.
(92) The compound or turmeric extract according to any one of (36) to (43), for treating or preventing a disease or symptom that is ameliorated by lowering blood glucose level, reducing blood hemoglobin A1c content, or increasing blood HDL-cholesterol content in the subject.

This description contains the contents disclosed in Japanese Patent Application No. 2019-122895, to which the present application claims priority.

### Advantageous Effects of Invention

One or more embodiments of the present invention provide a composition for lowering blood glucose level, reducing blood hemoglobin A1c content, or increasing blood HDL-cholesterol content.

One or more other embodiments of the present invention provide a composition for treating or preventing metabolic syndrome.

One or more still other embodiments of the present invention provide a composition for improving lipid metabolism or carbohydrate metabolism.

### BRIEF DESCRIPTION OF DRAWINGS

FIG. 1 shows amounts of change in blood glucose content in a group with intake of a test food comprising a turmeric extract (turmeric extract group, n=43) and those in a group with intake of a placebo food (placebo group, n=44) from before the intake to 4 weeks, 8 weeks, and 12 weeks after the intake.

FIG. 2 shows amounts of change in blood HbA1c content in a group with intake of a test food comprising a turmeric extract (turmeric extract group, n=43) and those in a group with intake of a placebo food (placebo group, n=44) from before the intake to 4 weeks, 8 weeks, and 12 weeks after the intake.

FIG. 3 shows blood HDL-cholesterol content in a group with intake of a test food comprising a turmeric extract (turmeric extract group, n=43) and that in a group with intake of a placebo food (placebo group, n=44) before the intake and 4 weeks, 8 weeks, and 12 weeks after the intake.

### DESCRIPTION OF EMBODIMENTS

### <Turmeric>

In one or more embodiments of the present invention, the term “turmeric” refers to a plant belonging to the genus Curcuma in the family Zingiberaceae, and specific examples thereof include Curcuma longa (autumn turmeric), Curcuma aromatica (spring turmeric), Curcuma zedoaria, Curcuma phaeocaulis, Curcuma kwangsiensis, Curcuma wenyujin, and/or Curcuma xanthorrhiza. Among these, Curcuma longa is particularly preferable.

In one or more embodiments of the present invention, use of a portion of turmeric including the rhizome is preferable.

The form of turmeric for use in one or more embodiments of the present invention is not limited, and may be a crushed product, squeezed juice, or extract of turmeric, or a processed product thereof. Particularly preferable is a turmeric extract. In the following, preferred embodiments of the turmeric extract will be described.

### <Turmeric Extract>

In one or more embodiments of the present invention, the term “turmeric extract” refers to an extract of a plant material derived from a plant of the genus Curcuma in the family Zingiberaceae, the extract being extracted with an extraction solvent. The turmeric extract is not limited to a solvent extract obtained via extraction with an extraction solvent, and the scope of the turmeric extract includes a resultant of additional fractionation or purification through column chromatography or the like for such a solvent extract. The turmeric extract for use in one or more embodiments of the present invention can be in the form of an extract solution after the completion of an extraction procedure (including a fractionation or purification procedure if fractionation or purification is performed), a concentrate obtained by partially removing the solvent from the extract solution, or a dry matter obtained by removing the solvent from the extract solution. The solvent can be removed from the extract by volatilizing the solvent via, for example, heating and/or decompressing. The methods for such heating and decompressing are not limited, and, for example, known methods can be employed.

Examples of the above plant material include the aforementioned turmeric rhizome. For example, a rhizome of Curcuma longa is particularly preferable. The rhizome for use may be one collected from soil. An adequate part of the rhizome in its original form, in pieces obtained by cutting into adequate dimensions or shape, or in the form of a grounded product may be used. The plant material may have been dried, as appropriate.

As an extraction solvent, water can be used. The scope of “water” includes hot water. As such hot water, for example, hot water with a temperature of 95° C. or higher can be used.

The extraction method to obtain the turmeric extract from the plant material is not limited.

In one or more embodiments of the present invention, use of a turmeric extract comprising at least one of turmeronol A, turmeronol B, and bisacurone and extracted with water is preferable, use of a turmeric extract comprising at least turmeronol A and turmeronol B and extracted with water is more preferable, and use of a turmeric extract comprising turmeronol A, turmeronol B, and bisacurone and extracted with water is further preferable.

Turmeronol A and turmeronol B are each a compound having a planar structure shown below.

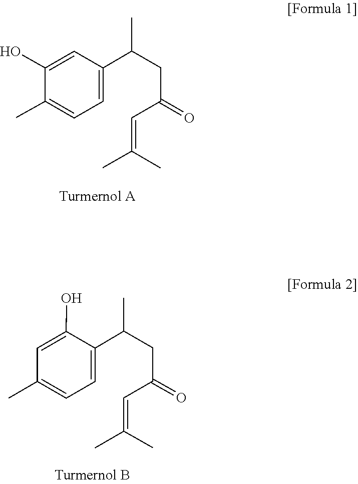 [SMILES: CC(C)=CC(=O)CC(C)c1ccc(C)c(O)c1 ; CC(C)=CC(=O)CC(C)c1ccc(C)cc1O]

In turmeronol A and turmeronol B in a naturally-occurring substance separated from turmeric, the configuration of the carbon at position 6 of the partial structure 2-methyl-2-hepten-4-one is S configuration. In one or more embodiments of the present invention, however, it is sufficient for each of turmeronol A and turmeronol B to have the corresponding planar structure shown above, and the configuration may be S configuration, R configuration, or a mixture of S and R configurations.

In one or more embodiments of the present invention, bisacurone is a compound having a planar structure shown below. Bisacurone has an asymmetric carbon at the position indicated with the symbol * in the formula of the planar structure, and there may be multiple optical isomers of bisacurone. In one or more embodiments of the present invention, bisacurone may be any optical isomer thereof, or a mixture of two or more optical isomers thereof.

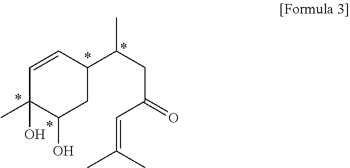 [SMILES: CC(C)=CC(=O)CC(C)C1C=CC(C)(O)C(O)C1]

### <Turmeronol A, Turmeronol B, and Bisacurone>

At least one of turmeronol A, turmeronol B, and bisacurone (also referred to as the “active compound”) for use in one or more embodiments of the present invention may be originated from a plant, or artificially synthesized. For example, optically active (+)-turmeronol A can be synthesized with a method described in Biosci Biotechnol Biochem. 1993; 57(7): 1137-40.

In one or more embodiments of the present invention, it is sufficient for each of turmeronol A, turmeronol B, and bisacurone to have the corresponding planar structure shown above. Each of turmeronol A, turmeronol B, and bisacurone may be any optical isomer thereof, or a mixture of a plurality of optical isomers thereof.

The active compound for use in one or more embodiments of the present invention is more preferably originated from a plant material, and further preferably originated from a plant of the genus Curcuma in the family Zingiberaceae. Specific examples of plants of the genus Curcuma in the family Zingiberaceae and parts thereof are as described above. The active compound can be obtained from a part, such as a rhizome, of a plant of the genus Curcuma in the family Zingiberaceae.

The active compound can be extracted from a plant material containing it. The extraction method is as described above. The active compound may be in the form of a plant extract, in particular, a turmeric extract extracted with water.

Alternatively, a fraction of the active compound highly purified from a plant extract containing the active compound may be used in one or more embodiments of the present invention, and such a fraction may be blended in the composition of one or more embodiments of the present invention. For example, the active compound can be highly purified in an ethyl acetate fraction by subjecting a plant extract containing the active compound to liquid-liquid distribution using ethyl acetate/water. Alternatively, a plant extract containing the active compound or a fraction thereof may be subjected to a purification process via chromatography to obtain the active compound as a highly purified product. Examples of chromatography techniques that can be employed include reversed-phase column chromatography and normal-phase thin-layer chromatography.

A plant extract containing the active compound or a fraction thereof may be subjected to processing, such as dehydration, pulverization, granulation, and solubilization, in accordance with a conventional technique.

The active compound preferably contains at least turmeronol A and turmeronol B, and more preferably contains turmeronol A, turmeronol B, and bisacurone.

<Composition for Lowering Blood Glucose Level, Reducing Blood Hemoglobin A1c Content, or Increasing Blood HDL-Cholesterol Content, Composition for Treating or Preventing Metabolic Syndrome, and Composition for Improving Lipid Metabolism or Carbohydrate Metabolism>

A first aspect of the composition of one or more embodiments of the present invention relates to a composition for lowering blood glucose level, reducing blood hemoglobin A1c (HbA1c) content, or increasing blood HDL-cholesterol content, wherein the composition comprises a turmeric extract comprising at least one of turmeronol A, turmeronol B, and bisacurone. The first aspect of the composition of one or more embodiments of the present invention has one or more effects, preferably two or more effects, more preferably three effects of lowering blood glucose level, reducing blood HbA1c content, and increasing blood HDL-cholesterol content. Each of blood glucose level, blood HbA1c content, and blood HDL-cholesterol content can be measured with a known method. For example, blood glucose level can be measured with an enzymatic method. Blood HbA1c content can be measured with high-performance liquid chromatography (HPLC). Blood HDL-cholesterol content can be measured with an enzymatic method.

It is preferable that blood glucose level, blood HbA1c content, and blood HDL-cholesterol content be each a measurement in fasting (in hunger).

In the first aspect of the composition of one or more embodiments of the present invention, “lowering blood glucose level” means lowering blood glucose level and/or inhibiting the increase of blood glucose level in a subject of administration.

In the first aspect of the composition of one or more embodiments of the present invention, “reducing blood hemoglobin A1c (HbA1c) content” means reducing blood HbA1c content and/or inhibiting the increase of blood HbA1c content in a subject of administration.

In the first aspect of the composition of one or more embodiments of the present invention, “increasing blood HDL-cholesterol content” means increasing blood HDL-cholesterol content and/or inhibiting the reduction of blood HDL-cholesterol content.

A second aspect of the composition of one or more embodiments of the present invention relates to a composition for treating or preventing metabolic syndrome, comprising at least one of turmeronol A, turmeronol B, and bisacurone. The second aspect of the composition of one or more embodiments of the present invention has an effect of treating or preventing metabolic syndrome.

A third aspect of the composition of one or more embodiments of the present invention relates to a composition for improving lipid metabolism or carbohydrate metabolism, comprising at least one of turmeronol A, turmeronol B, and bisacurone. The third aspect of the composition of one or more embodiments of the present invention has an effect of improving lipid metabolism or carbohydrate metabolism.

In the third aspect of the composition of one or more embodiments of the present invention, “improving lipid metabolism or carbohydrate metabolism” means improving lipid metabolism or carbohydrate metabolism and/or inhibiting the worsening of lipid metabolism or carbohydrate metabolism in a subject of administration.

The subject of the first, second, or third aspect of the composition of one or more embodiments of the present invention is typically a human, but is not limited to humans and may be any other non-human animal, such as a mammal other than humans. The subject of the first, second, or third aspect of the composition of one or more embodiments of the present invention may be a healthy subject, or a healthy or nonhealthy subject in need of the corresponding effect.

In one or more embodiments of the present invention, the subject is preferably a healthy subject, in particular, a healthy human. The healthy subject refers to such a subject that one or more, preferably two or more, more preferably all of the blood glucose level, blood HbA1c content, and blood HDL-cholesterol content are within the ranges of normal values, and, in the case of a human, refers to such a human that the blood glucose level, blood HbA1c content, and blood HDL-cholesterol content are within the following ranges of reference values. Non Patent Literature 8 discloses that, for humans, the reference value of fasting blood glucose level is 70 to 109 mg/dL, the reference value of blood HbA1c content is 4.6 to 6.2%, and the reference value of blood HDL cholesterol content is 40 to 70 mg/dL (male) or 45 to 75 mg/dL (female).

The first aspect of the composition of one or more embodiments of the present invention may be a composition for being ingested by or administering to a subject in need of lowering blood glucose level, reducing blood hemoglobin A1c content, or increasing blood HDL-cholesterol content or for whom lowering blood glucose level, reducing blood hemoglobin A1c content, or increasing blood HDL-cholesterol content is desirable. Examples of the subject include a subject whose blood glucose level is higher than normal values, a subject whose blood HbA1c content is higher than normal values, and a subject whose blood HDL-cholesterol content is lower than normal values. Even in the case of a subject such that one or more, preferably two or more, more preferably all of the blood glucose level, blood HbA1c content, and blood HDL-cholesterol content are within the ranges of normal values, if the subject is a subject for whom inhibiting the increase of blood glucose level, inhibiting the increase of blood HbA1c content, or inhibiting the reduction of blood HDL-cholesterol content is desirable, the subject corresponds to the above “subject for whom lowering blood glucose level, reducing blood HbA1c content, or increasing blood HDL-cholesterol content is desirable”. The first aspect of the composition of one or more embodiments of the present invention is preferably used for treating or preventing a disease or symptom that is ameliorated by lowering blood glucose level, reducing blood HbA1c content, or increasing blood HDL-cholesterol content.

The second aspect of the composition of one or more embodiments of the present invention may be a composition for being ingested by or administering to a subject in need of treating or preventing metabolic syndrome or for whom treating or preventing metabolic syndrome is desirable. Examples of the subject include a subject affected by or suspected to be affected by metabolic syndrome, and a subject for whom preventing metabolic syndrome is desirable in spite that the subject is healthy.

The third aspect of the composition of one or more embodiments of the present invention may be a composition for being ingested by or administering to a subject in need of improving lipid metabolism or carbohydrate metabolism or for whom improving lipid metabolism or carbohydrate metabolism is desirable. Examples of the subject include a subject whose blood glucose level is higher than normal values, a subject whose blood HbA1c content is higher than normal values, a subject such that another index of carbohydrate metabolism deviates from normal values, a subject whose blood HDL-cholesterol content is lower than normal values, a subject such that another index of lipid metabolism deviates from normal values, and a subject affected by or suspected to be affected by metabolic syndrome. Even in the case of a subject such that one or more, preferably two or more, more preferably all of the blood glucose level, blood HbA1c content, blood HDL-cholesterol content, and other indexes of carbohydrate metabolism or lipid metabolism are within the ranges of normal values or in the case of a subject not affected by metabolic syndrome, if the subject is a subject for whom inhibiting the worsening of lipid metabolism or carbohydrate metabolism is desirable, the subject corresponds to the above “subject for whom improving lipid metabolism or carbohydrate metabolism is desirable”. The third aspect of the composition of one or more embodiments of the present invention is preferably used for treating or preventing a disease or symptom that is ameliorated by improving lipid metabolism or carbohydrate metabolism.

In the present description, the first aspect, second aspect, and third aspect of the composition of one or more embodiments of the present invention are collectively expressed as “the composition of one or more embodiments of the present invention”.

The composition of one or more embodiments of the present invention may be a composition in the form of, for example, a pharmaceutical product, a food or beverage product, a feed, a food additive, or a feed additive, and is preferably in the form of a pharmaceutical product or a food or beverage product. Food or beverage products include forms of foods with functional claims, foods for specified health use, and nutritional supplements. The composition of one or more embodiments of the present invention may be a composition for use in medical applications (e.g., a pharmaceutical product), or a composition for use in nonmedical applications (e.g., a food or beverage product, a feed, a food additive, a feed additive).

The composition of one or more embodiments of the present invention is preferably in the form of a composition that is ingested or administered via oral or transnasal administration, and is more preferably in the form of a composition that is ingested or administered via oral administration.

In one or more embodiments of the present invention, the term “daily intake” is used to mean the total amount of the composition of one or more embodiments of the present invention that is ingested or administered per day, and is used to preferably mean the total amount of the composition of one or more embodiments of the present invention that is ingested by or administered to one human, in particular, one adult human, per day. A specific example of the “daily intake” of the composition of one or more embodiments of the present invention when ingested or administered via oral or transnasal administration, preferably via oral administration, is 0.1 g to 500 g as the amount of the composition of one or more embodiments of the present invention. The composition of one or more embodiments of the present invention may be ingested or administered continuously, or ingested or administered when needed.

The shape of the composition of one or more embodiments of the present invention is not limited, and may be, for example, any shape such as liquid, fluid, gel, semi-solid, and solid.

The composition of one or more embodiments of the present invention may further comprise at least one additional component, in addition to the above active compound or the above turmeric extract. Preferable examples of the at least one additional component that may be comprised in the composition of one or more embodiments of the present invention include, but are not limited to, components that are acceptable in the final form, such as a pharmaceutical product, a food or beverage product, a feed, a food additive, and a feed additive, and are orally ingestible.

Examples of the additional component include sweeteners, acidulants, vitamins, minerals, thickeners, emulsifiers, antioxidants, and water. According to need, a pigment, an aroma chemical, a preservative, an antiseptic agent, a fungicide, or an additional physiologically active substance or the like may be added.

Examples of sweeteners include: monosaccharides and disaccharides, such as glucose, fructose, sucrose, lactose, maltose, palatinose, trehalose, and xylose; isomerized glucose syrup (e.g., glucose-fructose syrup, fructose-glucose syrup, and an isomerized sugar mixture), sugar alcohols (e.g., erythritol, xylitol, lactitol, Palatinit, sorbitol, and reduced starch syrup), honey, and high-intensity sweeteners (e.g., sucralose, acesulfame potassium, thaumatin, stevia, and aspartame).

Examples of acidulants include citric acid, malic acid, gluconic acid, tartaric acid, lactic acid, phosphoric acid, and salts thereof. One of these or two or more thereof can be used.

Examples of vitamins include vitamin A, vitamin B1, vitamin B2, vitamin B6, vitamin E, niacin, and inositol.

Examples of minerals include calcium, magnesium, zinc, and iron.

Examples of thickeners include carrageenan, gellan gum, xanthan gum, gum Arabic, tamarind gum, guar gum, Locust bean gum, karaya gum, agar, gelatin, pectin, soybean polysaccharides, and carboxymethyl cellulose (CMC).

Examples of emulsifiers include glycerin fatty acid ester, sucrose fatty acid ester, sorbitan fatty acid ester, lecithin, plant sterol, and saponin.

Examples of antioxidants include vitamin C, tocopherol (vitamin E), and enzyme-treated rutin.

The additional components can be adequately blended in such amounts that those skilled in the art generally employ for compositions for food or beverage products, pharmaceutical products, or others.

The composition of one or more embodiments of the present invention preferably comprises only the active compound or the turmeric extract as an active component involved in the intended activity in each aspect of the composition of one or more embodiments of the present invention. In this case, the composition of one or more embodiments of the present invention may comprise any additional component, as described above, that is not involved in the intended activity in each aspect of the composition of one or more embodiments of the present invention.

In one or more embodiments of the present invention, the form of the composition formulated with the above active compound or the above turmeric extract and at least one additional component by an adequate means may be the form of any of solid compositions including powders, granules, capsules, and tablets (including coated tablets such as sugar-coated tablets, multilayer tablets, orally disintegrating tablets, and chewable tablets), or the form of any of liquid compositions including solutions.

The content of the active compound or the turmeric extract comprising it in the composition of one or more embodiments of the present invention is not limited, and may be any content effective for exerting the intended activity in each aspect.

The composition of one or more embodiments of the present invention comprises preferably 20 μg or more, more preferably 37 μg or more, particularly preferably 55 μg or more, further preferably 74 μg or more, most preferably 100 μg or more of turmeronol A and turmeronol B in total per daily intake. In this case, the first aspect of the composition of one or more embodiments of the present invention has particularly high effect of lowering blood glucose level, reducing blood hemoglobin A1c content, or increasing blood HDL-cholesterol content, the second aspect of the composition of one or more embodiments of the present invention has particularly high effect of treating or preventing metabolic syndrome, and the third aspect of the composition of one or more embodiments of the present has particularly high effect of improving lipid metabolism or carbohydrate metabolism. The upper limit of the total content of turmeronol A and turmeronol B is not limited, and the composition of one or more embodiments of the present invention comprises typically 1100 μg or less, preferably 550 μg or less, more preferably 330 μg or less, particularly preferably 220 μg or less, further preferably 170 μg or less, most preferably 125 μg or less of turmeronol A and turmeronol B in total per daily intake. If at least one of turmeronol A and turmeronol B in the composition of one or more embodiments of the present invention is in the form of a turmeric extract comprising it, it is preferable for the composition to comprise the turmeric extract in such a manner that the total amount of turmeronol A and turmeronol B falls within the above range.

If the composition of one or more embodiments of the present invention comprises a turmeric extract comprising at least one of turmeronol A, turmeronol B, and bisacurone, the turmeric extract in the composition may comprise preferably 20 μg or more, more preferably 37 μg or more, particularly preferably 55 μg or more, further preferably 74 μg or more, most preferably 100 μg or more of turmeronol A and turmeronol B in total per daily intake. The upper limit of the total content of turmeronol A and turmeronol B in the turmeric extract is not limited, and the total content may be 1100 μg or less, preferably 550 μg or less, more preferably 330 μg or less, particularly preferably 220 μg or less, further preferably 170 μg or less, and most preferably 125 μg or less per daily intake.

The composition of one or more embodiments of the present invention comprises preferably 17 μg or more, more preferably 28 μg or more, particularly preferably 43 μg or more, further preferably 57 μg or more, most preferably 77 μg or more of turmeronol A per daily intake. In this case, the first aspect of the composition of one or more embodiments of the present invention has particularly high effect of lowering blood glucose level, reducing blood hemoglobin A1c content, or increasing blood HDL-cholesterol content, the second aspect of the composition of one or more embodiments of the present invention has particularly high effect of treating or preventing metabolic syndrome, and the third aspect of the composition of one or more embodiments of the present has particularly high effect of improving lipid metabolism or carbohydrate metabolism. The upper limit of the content of turmeronol A is not limited, and the composition of one or more embodiments of the present invention comprises typically 900 μg or less, preferably 430 μg or less, more preferably 260 μg or less, particularly preferably 175 μg or less, further preferably 130 μg or less, most preferably 95 μg or less of turmeronol A per daily intake. If turmeronol A in the composition of one or more embodiments of the present invention is in the form of a turmeric extract comprising it, it is preferable for the composition to comprise the turmeric extract in such a manner that the amount of turmeronol A falls within the above range.

If the composition of one or more embodiments of the present invention comprises a turmeric extract comprising at least one of turmeronol A, turmeronol B, and bisacurone, the turmeric extract in the composition may comprise preferably 17 μg or more, more preferably 28 μg or more, particularly preferably 43 μg or more, further preferably 57 μg or more, most preferably 77 μg or more of turmeronol A per daily intake. The upper limit of the content of turmeronol A in the turmeric extract is not limited, and the content may be typically 900 μg or less, preferably 430 μg or less, more preferably 260 μg or less, particularly preferably 175 μg or less, further preferably 130 μg or less, and most preferably 95 μg or less per daily intake.

The composition of one or more embodiments of the present invention comprises preferably 5 μg or more, more preferably 8 μg or more, particularly preferably 12 μg or more, further preferably 16 μg or more, most preferably 22 μg or more of turmeronol B per daily intake. In this case, the first aspect of the composition of one or more embodiments of the present invention has particularly high effect of lowering blood glucose level, reducing blood hemoglobin A1c content, or increasing blood HDL-cholesterol content, the second aspect of the composition of one or more embodiments of the present invention has particularly high effect of treating or preventing metabolic syndrome, and the third aspect of the composition of one or more embodiments of the present has particularly high effect of improving lipid metabolism or carbohydrate metabolism. The upper limit of the content of turmeronol B is not limited, and the composition of one or more embodiments of the present invention comprises typically 250 μg or less, preferably 123 μg or less, more preferably 74 μg or less, particularly preferably 50 μg or less, further preferably 37 μg or less, most preferably 27 μg or less of turmeronol B per daily intake. If turmeronol B in the composition of one or more embodiments of the present invention is in the form of a turmeric extract comprising it, it is preferable for the composition to comprise the turmeric extract in such a manner that the amount of turmeronol B falls within the above range.

If the composition of one or more embodiments of the present invention comprises a turmeric extract comprising at least one of turmeronol A, turmeronol B, and bisacurone, the turmeric extract in the composition may comprise preferably 5 μg or more, more preferably 8 μg or more, particularly preferably 12 μg or more, further preferably 16 μg or more, most preferably 22 μg or more of turmeronol B per daily intake. The upper limit of the content of turmeronol B in the turmeric extract is not limited, and the content may be typically 250 μg or less, preferably 123 μg or less, more preferably 74 μg or less, particularly preferably 50 μg or less, further preferably 37 μg or less, and most preferably 27 μg or less per daily intake.

The composition of one or more embodiments of the present invention comprises preferably 80 μg or more, more preferably 133 μg or more, particularly preferably 200 μg or more, further preferably 267 μg or more, most preferably 360 μg or more of bisacurone per daily intake. In this case, the first aspect of the composition of one or more embodiments of the present invention has particularly high effect of lowering blood glucose level, reducing blood hemoglobin A1c content, or increasing blood HDL-cholesterol content, the second aspect of the composition of one or more embodiments of the present invention has particularly high effect of treating or preventing metabolic syndrome, and the third aspect of the composition of one or more embodiments of the present has particularly high effect of improving lipid metabolism or carbohydrate metabolism. The upper limit of the content of bisacurone is not limited, and the composition of one or more embodiments of the present invention comprises typically 4000 μg or less, preferably 2000 μg or less, more preferably 1200 μg or less, particularly preferably 800 μg or less, further preferably 600 μg or less, most preferably 440 μg or less of bisacurone per daily intake. If bisacurone in the composition of one or more embodiments of the present invention is in the form of a turmeric extract comprising it, it is preferable for the composition to comprise the turmeric extract in such a manner that the amount of bisacurone falls within the above range.

If the composition of one or more embodiments of the present invention comprises a turmeric extract comprising at least one of turmeronol A, turmeronol B, and bisacurone, the turmeric extract in the composition may comprise preferably 80 μg or more, more preferably 133 μg or more, particularly preferably 200 μg or more, further preferably 267 μg or more, most preferably 360 μg or more of bisacurone per daily intake. The upper limit of the content of bisacurone in the turmeric extract is not limited, and the content may be typically 4000 μg or less, preferably 2000 μg or less, more preferably 1200 μg or less, particularly preferably 800 μg or less, further preferably 600 μg or less, and most preferably 440 μg or less per daily intake.

In the composition of one or more embodiments of the present invention, the content of curcumin is typically less than 30 mg, preferably 5 mg or less, more preferably 2.5 mg or less, particularly preferably 1.5 mg or less, further preferably 1 mg or less, still further preferably 700 μg or less, and most preferably 520 μg or less per daily intake. In this case, the first aspect of the composition of one or more embodiments of the present invention has particularly high effect of lowering blood glucose level, reducing blood hemoglobin A1c content, or increasing blood HDL-cholesterol content, the second aspect of the composition of one or more embodiments of the present invention has particularly high effect of treating or preventing metabolic syndrome, and the third aspect of the composition of one or more embodiments of the present has particularly high effect of improving lipid metabolism or carbohydrate metabolism.

If the composition of one or more embodiments of the present invention comprises a turmeric extract comprising at least one of turmeronol A, turmeronol B, and bisacurone, the content of curcumin in the turmeric extract in the composition may be typically less than 30 mg, preferably 5 mg or less, more preferably 2.5 mg or less, particularly preferably 1.5 mg or less, further preferably 1 mg or less, furthermore preferably 700 μg or less, and most preferably 520 μg or less per daily intake.

The composition of one or more embodiments of the present invention is preferably a composition that is continuously ingested, specifically, a composition that is continuously ingested at a frequency of once or twice or more per day, preferably over 4 weeks or more, more preferably over 8 weeks or more, most preferably over 12 weeks or more.

The composition of one or more embodiments of the present invention may be the above active compound or the above turmeric extract comprising it, per se, or a composition comprising the above active compound or the above turmeric extract comprising it and at least one additional component. When the composition of one or more embodiments of the present invention comprises the above active compound or the above turmeric extract comprising it and at least one additional component, the composition may be a composition obtained by mixing the above active compound or the above turmeric extract comprising it and at least one additional component, or a composition obtained by formulating with the active compound or the turmeric extract comprising it and at least one additional component by an adequate means, or a composition obtained by mixing a composition obtained by formulating with the active compound or the turmeric extract comprising it and at least one additional component with another additional component.

### <Method for Lowering Blood Glucose Level, Reducing Blood Hemoglobin A1c Content, or Increasing Blood HDL-Cholesterol Content, Method for Treating or Preventing Metabolic Syndrome, and Method for Improving Lipid Metabolism or Carbohydrate Metabolism>

A first aspect of the method of one or more embodiments of the present invention relates to a method for lowering blood glucose level, reducing blood hemoglobin A1c content, or increasing blood HDL-cholesterol content, comprising:

administering at least one of turmeronol A, turmeronol B, and bisacurone, or a turmeric extract comprising at least one of turmeronol A, turmeronol B, and bisacurone and extracted with water, to a subject; and lowering blood glucose level, reducing blood hemoglobin A1c content, or increasing blood HDL-cholesterol content in the subject.

The subject in the first aspect is a subject in need of lowering blood glucose level, reducing blood hemoglobin A1c content, or increasing blood HDL-cholesterol content or for whom lowering blood glucose level, reducing blood hemoglobin A1c content, or increasing blood HDL-cholesterol content is desirable. The method according to the first aspect is preferably a method for treating or preventing a disease or symptom that is ameliorated by lowering blood glucose level, reducing blood hemoglobin A1c content, or increasing blood HDL-cholesterol content in the subject.

A second aspect of the method of one or more embodiments of the present invention relates to a method for treating or preventing metabolic syndrome, comprising:

administering at least one of turmeronol A, turmeronol B, and bisacurone, or a turmeric extract comprising at least one of turmeronol A, turmeronol B, and bisacurone and extracted with water, to a subject; and

treating or preventing metabolic syndrome in the subject.

The subject in the second aspect is a subject in need of treating or preventing metabolic syndrome or for whom treating or preventing metabolic syndrome is desirable.

A third aspect of the method of one or more embodiments of the present invention relates to a method for improving lipid metabolism or carbohydrate metabolism, comprising:

administering at least one of turmeronol A, turmeronol B, and bisacurone, or a turmeric extract comprising at least one of turmeronol A, turmeronol B, and bisacurone and extracted with water, to a subject; and

improving lipid metabolism or carbohydrate metabolism in the subject.

The subject in the third aspect is a subject in need of improving lipid metabolism or carbohydrate metabolism or for whom improving lipid metabolism or carbohydrate metabolism is desirable. The method according to the third aspect is preferably a method for treating or preventing a disease or symptom that is ameliorated by improving lipid metabolism or carbohydrate metabolism.

The active compound or the turmeric extract for use in each of the above aspects of the method of one or more embodiments of the present invention can be in the aforementioned form of the composition of one or more embodiments of the present invention.

In each of the above aspects of the method of one or more embodiments of the present invention, the meaning of “administering” the active compound or the turmeric extract to the subject encompasses that the subject voluntarily ingests the active compound or the turmeric extract, and that the active compound or the turmeric extract is administered to or ingested by the subject in compliance with instructions of a physician or a veterinarian.

Each of the above aspects of the method of one or more embodiments of the present invention may be a medical method or a nonmedical method.

The subject in each of the above aspects of the method of one or more embodiments of the present invention is typically a human, but may be a mammal other than humans. Examples of the subject in the first aspect of the method of one or more embodiments of the present invention can include a subject whose blood glucose level is higher than normal values, a subject whose blood HbA1c content is higher than normal values, a subject whose blood HDL-cholesterol content is lower than normal values, and a subject such that one or more, two or more, or all of the blood glucose level, blood HbA1c content, and blood HDL-cholesterol content are within the ranges of normal values. Examples of the subject in the second aspect of the method of one or more embodiments of the present invention can include a subject affected by or suspected to be affected by metabolic syndrome, and a subject not affected by metabolic syndrome. Examples of the subject in the third aspect of the method of one or more embodiments of the present invention can include a subject whose blood glucose level is higher than normal values, a subject whose blood HbA1c content is higher than normal values, a subject such that another index of carbohydrate metabolism deviates from normal values, a subject whose blood HDL-cholesterol content is lower than normal values, a subject such that another index of lipid metabolism deviates from normal values, a subject affected by or suspected to be affected by metabolic syndrome, a subject such that one or more, two or more, or all of the blood glucose level, blood HbA1c content, blood HDL-cholesterol content, and other indexes of carbohydrate metabolism or lipid metabolism are within the ranges of normal values, and a subject not affected by metabolic syndrome.

In each of the aspects of the method of one or more embodiments of the present invention, the administration route is preferably oral or transnasal administration, and is particularly preferably oral administration.

In each of the aspects of the method of one or more embodiments of the present invention, the amount of the active compound or the turmeric extract administered to the subject is not limited, and may be any amount effective for allowing the intended effect in each aspect to be exerted. For example, in one embodiment, in the case that the subject is an adult human, it is preferable to administer the active compound or the turmeric extract to the subject, so that 20 μg or more of turmeronol A and turmeronol B in total can be administered to the subject per day. In another embodiment, in the case that the subject is an adult human, it is preferable to administer the active compound or the turmeric extract to the subject, so that 17 μg or more of turmeronol A can be administered to the subject per day. In another embodiment, in the case that the subject is an adult human, it is preferable to administer the active compound or the turmeric extract to the subject, so that 5 μg or more of turmeronol B can be administered to the subject per day. In another embodiment, in the case that the subject is an adult human, it is preferable to administer the active compound or the turmeric extract to the subject, so that 80 μg or more of bisacurone can be administered to the subject per day. Further, it is preferable to administer the active compound or the turmeric extract to the subject, so that the daily dose of curcumin can be less than 30 mg. Further preferably, it is preferable to administer the active compound or the turmeric extract to the subject, so that each component of the active compound can be administered to the subject per day at a weight that is described as a preferred amount for the component of the active compound in the daily intake of the composition of one or more embodiments of the present invention.

### EXAMPLES

### 1. Method for Production of Turmeric Extract

A turmeric extract was prepared by extracting the rhizome part of turmeric (Curcuma longa) with water, and heat-drying the resulting extract under reduced pressure to remove water. The amounts of turmeronol A (TA) and turmeronol B (TB) in the turmeric extract were measured by using LC/MS, and the amounts of bisacurone and curcumin were measured by using HPLC.

### 2. Test Food

The test food was a tablet comprising the turmeric extract containing, per three tablets, 86.5 μg of TA, 24.7 μg of TB, 400 μg of bisacurone, and 471 μg of curcumin. The tablet was prepared by mixing the turmeric extract, an excipient, a lubricant, a coloring agent, and other ingredients for manufacturing (fine grain silicon dioxide and sucrose fatty acid ester), and tableting the mixture.

The placebo food was a tablet prepared with replacing the turmeric extract in the test food with an excipient.

### 3. Method for Measuring Glucose, HbA1c, and HDL-Cholesterol Contents in Blood

Blood glucose content was measured with an enzymatic method, blood HbA1c content was measured with high-performance liquid chromatography (HPLC), and blood HDL-cholesterol content was measured with an enzymatic method.

### 4. Evaluation Test for Glucose, HbA1c, and HDL-Cholesterol Contents in Blood

Eighty-seven healthy 50- to 69-year-old male and female test subjects were randomly assigned to two groups (placebo group: 44 test subjects, turmeric extract group: 43 test subjects), and, under a double-blind test, allowed to ingest three tablets of the test food, which contained the turmeric extract, or the placebo food, which did not contain the extract, once a day before supper for 12 weeks. Contents of glucose, HbA1c, and HDL cholesterol in blood were measured for the test subjects before the intake of the test food or the placebo food, and 4 weeks, 8 weeks, and 12 weeks after the intake. The test subjects were fasted, but allowed to drink water, during a period between 9:00 p.m. on the day before the measurement and the measurement on the following day. Thus, blood glucose, blood HbA1c, and blood HDL cholesterol in fasting were measured.

Before intake of the test food or the placebo food (on the day of initiation of intake), blood glucose (mean±standard deviation) was 84.2±6.0 mg/dL in the placebo group and 86.0±6.9 mg/dL in the turmeric extract group, blood HbA1c (mean±standard deviation) was 5.48±0.24% in the placebo group and 5.51±0.28% in the turmeric extract group, and blood HDL cholesterol (mean±standard deviation) was 54.3±14.9 mg/dL in the placebo group and 56.0±12.3 mg/dL in the turmeric extract group. As described in Non Patent Literature 8, the reference value of blood glucose is 70 to 109 mg/dL, the reference value of blood HbA1c is 4.6 to 6.2%, and the reference value of blood HDL cholesterol is 40 to 70 mg/dL (male) or 45 to 75 mg/dL (female). In the present experiment, the test subjects were healthy humans.

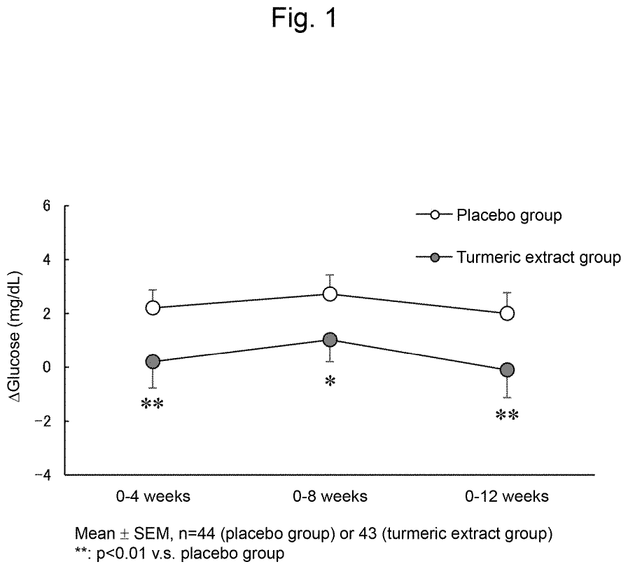

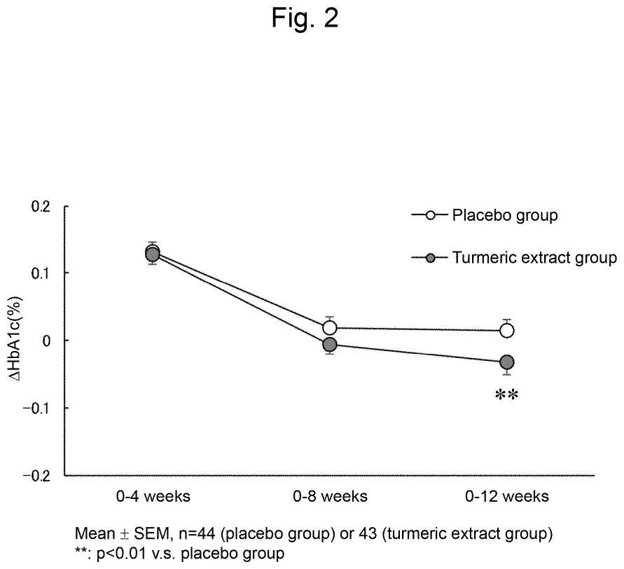

FIGS. 1 and 2 respectively show amounts of change in content of glucose in blood (mg/dL) and amounts of change in content of HbA1c in blood (%) from before the intake of the test food or the placebo food to 4 weeks, 8 weeks, and 12 weeks after the intake. As demonstrated in FIG. 1, the blood glucose contents in the turmeric extract group with intake of the turmeric extract-containing test food 4 weeks, 8 weeks, and 12 weeks after the initiation of intake of the test food were all significantly lower than those in the placebo group with intake of the placebo food. As demonstrated in FIG. 2, the blood HbA1c contents in the turmeric extract group 12 weeks after the initiation of intake of the test food were all significantly lower than those in the placebo group.

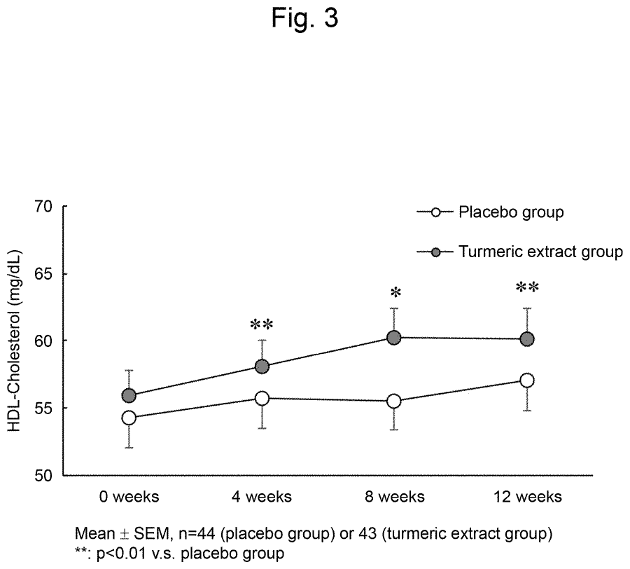

FIG. 3 shows measurement results for content of HDL-cholesterol in blood (mg/dL) before the intake of the test food or the placebo food, and 4 weeks, 8 weeks, and 12 weeks after the intake. As demonstrated in FIG. 3, the contents of HDL-cholesterol in blood in the turmeric extract group 4 weeks, 8 weeks, and 12 weeks after the intake were significantly higher than those in the placebo group.

These results revealed that intake of the turmeric extract decreases contents of glucose and HbA1c in blood, and increases content of HDL-cholesterol in blood.

All the publications, patents, and patent applications cited in the present description are incorporated herein by reference in their entirety.

1-9. (canceled)

10: A method for lowering blood glucose level, reducing blood hemoglobin A1c content, or increasing blood HDL-cholesterol content, comprising:
administering at least one of turmeronol A, turmeronol B, to a subject; and
lowering blood glucose level, reducing blood hemoglobin A1c content, or increasing blood HDL-cholesterol content in the subject.

11: The method according to claim 10, wherein the administering comprises administering a turmeric extract comprising at least one of turmeronol A, turmeronol B, and bisacurone and extracted with water, to the subject.

12: The method according to claim 10, wherein the subject is in need of lowering blood glucose level, reducing blood hemoglobin A1c content, or increasing blood HDL-cholesterol content or a subject for whom lowering blood glucose level, reducing blood hemoglobin A1c content, or increasing blood HDL-cholesterol content is desirable.

13: The method according to claim 10, wherein the subject is a healthy human.

14: The method according to claim 1, wherein the administering comprises 20 μg or more of turmeronol A and turmeronol B in total to the subject per day.

15: The method according to claim 10, wherein the administering comprises 17 μg or more of turmeronol A to the subject per day.

16: The method according to claim 10, wherein the administering comprises 5 μg or more of turmeronol B to the subject per day.

17: The method according to claim 10, wherein the administering comprises 80 μg or more of bisacurone to the subject per day.

18: The method according to claim 10, wherein the administering comprises administering the at least one of turmeronol A, turmeronol B, and bisacurone, so that the daily dose of curcumin is less than 30 mg.

19: A method for treating or preventing metabolic syndrome, comprising:
administering at least one of turmeronol A, turmeronol B, and bisacurone, or a turmeric extract comprising at least one of turmeronol A, turmeronol B, and bisacurone and extracted with water, to a subject; and
treating or preventing metabolic syndrome in the subject.

20: The method according to claim 19, wherein the administering comprises administering a turmeric extract comprising at least one of turmeronol A, turmeronol B, and bisacurone and extracted with water, to the subject.

21: The method according to claim 19, wherein the subject is in need of treating or preventing metabolic syndrome or a subject for whom treating or preventing metabolic syndrome is desirable.

22: The method according to claim 19, wherein the subject is a healthy human.

23: The method according to claim 19, wherein the administering comprises 20 μg or more of turmeronol A and turmeronol B in total to the subject per day.

24: The method according to claim 19, wherein the administering comprises 17 μg or more of turmeronol A to the subject per day.

25: The method according to claim 19, wherein the administering comprises 5 μg or more of turmeronol B to the subject per day.

26: The method according to claim 19, wherein the administering comprises 80 μg or more of bisacurone to the subject per day.

27: The method according to claim 19, wherein the administering comprises administering the at least one of turmeronol A, turmeronol B, and bisacurone, so that the daily dose of curcumin is less than 30 mg.

28: A method for improving lipid metabolism or carbohydrate metabolism, comprising:
administering at least one of turmeronol A, turmeronol B, and bisacurone, or a turmeric extract comprising at least one of turmeronol A, turmeronol B, and bisacurone and extracted with water, to a subject; and
improving lipid metabolism or carbohydrate metabolism in the subject.

29: The method according to claim 28, wherein the administering comprises administering a turmeric extract comprising at least one of turmeronol A, turmeronol B, and bisacurone and extracted with water, to the subject.

30: The method according to claim 28, wherein the subject is in need of improving lipid metabolism or carbohydrate metabolism or a subject for whom improving lipid metabolism or carbohydrate metabolism is desirable.

31: The method according to claim 28, wherein the subject is a healthy human.

32: The method according to claim 28, wherein the administering comprises 20 μg or more of turmeronol A and turmeronol B in total to the subject per day.

33: The method according to claim 28, wherein the administering comprises 17 μg or more of turmeronol A to the subject per day.

34: The method according to claim 28, wherein the administering comprises 5 μg or more of turmeronol B to the subject per day.

35: The method according to claim 28, wherein the administering comprises 80 μg or more of bisacurone to the subject per day.

36: The method according to claim 28, wherein the administering comprises administering the at least one of turmeronol A, turmeronol B, and bisacurone, so that the daily dose of curcumin is less than 30 mg.
